(19) United States
(12) Patent Application Publication
(10) Pub. No.: US 2008/0020025 A1
(43) Pub. Date: Jan. 24, 2008

(54) COMPOSITION FOR WOUND CARE AND METHOD OF USING SAME

(75) Inventor: Brian C. Giles, Scottsdale, AZ (US)

Correspondence Address: SNELL & WILMER L.L.P. (Main), 400 EAST VAN BUREN, ONE ARIZONA CENTER, PHOENIX, AZ, 85004-2202, US

(73) Assignee: PHARMAIONX, INC., Scottsdale, AZ (US)

(21) Appl. No.: 11/757,064

(22) Filed: Jun. 1, 2007

Related U.S. Application Data
(63) Continuation-in-part of application No. 10/469,568, filed on Aug. 28, 2003, now abandoned.
(63) Continuation-in-part of application No. 10/867,115, filed on Jun. 14, 2004, now abandoned.
(63) Continuation-in-part of application No. 11/753,547, filed on May 24, 2007.
(60) Provisional application No. 60/809,984, filed on Jun. 1, 2006.
(60) Provisional application No. 60/879,857, filed on Jan. 11, 2007.

(51) Int. Cl. A61K 33/42; A61K 33/14; A61L 15/16; A61P 31/02; A61P 17/02; A61K 33/20

(52) U.S. Cl. 424/446; 424/601; 424/663; 424/722

(57) ABSTRACT

This invention encompasses an expeditious method and composition for treating a wide variety of wounds and injuries by promoting the elevation of pH at the site of injury, enhancing the reduction or elimination of pain and scaring from the patho-physiological process of infection proliferation, and promoting enhanced wound care management and wound healing. The invention also provides a method of treating a subject having a condition associated with the presence of free radicals in quantities sufficient to cause undesirable symptoms. The invention encompasses controlling mechanisms of intracellular and extra-cellular ionic physiology through the administration of a composition which includes alkaline salts preferably cesium salts, for pH modulating and for restoring and enhancing the localized cellular ionic physiology. The composition promotes the increase of the electrical current density to certain types of wounds and also functions to treat and/or prevent adhesions resulting from surgeries. The composition simultaneously normalizes the localized ionic environment, thereby normalizing immune responses and reducing acidotic induced pain.

### CROSS-REFERENCE TO RELATED APPLICATIONS

This application is a continuation-in-part patent application of Ser. No. 10/469,568 filed Feb. 28, 2001, Ser. No. 10/867,115 filed Jun. 14, 2004, and Ser. No. 11/753,547 filed May 24, 2007, and also claims priority to U.S. Provisional Patent Application having Ser. No. 60/809,984 filed Jun. 1, 2006 and U.S. Provisional Patent Application having Ser. No. 60/879,857 filed Jan. 11, 2007, all of which are herein incorporated in their entirety.

### FIELD OF INVENTION

The present invention generally relates to compositions for use in medicine, e.g. as topical solutions, infusions, or surgical rinse solutions, and to processes for their preparation. The compositions of the invention include cesium and or rubidium salts and surfactants.

More particularly, the present invention relates to a method for woundcare management and a method for providing anti-scarring, anti-microbial, anti-inflammatory and anti-pain compositions and wound dressings. The compositions of the present invention may be used in combination with bandages, other solutions, or other topically applied materials. The invention also relates to a method of treating burns and wounds and the use of the subject compositions as antimicrobial agents.

### BACKGROUND OF THE INVENTION

It has been recognized for some time that inflammation in mammalian species can be traced at least in part to active oxygen species, including super-oxides, and radicals associated at the inflammatory site. The present invention provides compounds and methods for the treatment of subjects having inflammatory conditions or other conditions associated with free radicals which eliminate prior art drawbacks. The invention provides a method and composition for treating a subject having conditions resulting from active oxygen or super-oxide toxicity, which may result in acute or chronic inflammation or other disorders, such as arthritis.

Treating first, second, and third degree burns has long been, and still remains, one of the most difficult medical problems. The criteria for success of any method for treating a burn includes proper contraction of the wound, epithelialization, hair follicle preservation and restoration, and the assessment of newly formed granulation tissue. Contraction represents the difference between the initial wound size of the burn and the size of the burn twelve days later (12th post burn day or PBD), which includes both open and healed areas calculated as a percentage of the initial wound size. Epithelialization represents the percentage of the newly covered areas of the burn surface on the 12th PBD out of the total wound area on that same day. The presence of hair follicles indicates maintenance or restoration of dermal microcirculation and prevention of tissue ischemia and postischemic damage. The preservation of hair follicles and their count should be carried out microscopically in tissue sections. Additionally, it is important to assess newly formed granulation tissue when evaluating burn treatment protocols. Thickness of the new collagen layer during burn healing should be measured on 12^{th} PBD.

The invention also encompasses a composition and method for treating burns and wounds. The compounds of the present invention may be used as an antibacterial agent that can help prevent the colonization of the wound by pathologic agents. The method of the present invention maximizes epithelialization of the burn on a macroscopic level and maximizes hair follicle preservation on a microscopic level.

Animal cell plasma membranes contain four major phospholipids that represent greater than half of the total lipid: phosphatidylethanolamine, phosphatidylcholine, phosphatidylserine and sphingomyelin. Phosphatidylcholine and sphingomyelin are found mostly in the outer leaflet, while phosphatidylethanolamine and phosphatidylserine are found principally in the inner leaflet. The predominance of the negatively charged phosphatidylserine and phosphatidylinositol in the outer leaflet results in a net negative charge on the cell surface. Plasma membranes help maintain cellular integrity and are selectively permeable. While some molecules are able to diffuse through membranes, most, including ATP, require other means to enter such as transport proteins or channels. While glycolysis can provide energy to cells, the supply is limited because the cellular environment becomes acidic or acidotic, injuring or killing the cell and inhibiting ATP production.

The prior art includes numerous examples of incorporating biologically active substances, such as antibiotics and antiseptics, in the continuous phase with adhesive bandages. The prior art discloses incorporating a biologically active substance in a separate phase, which is dispersed in the continuous phase, thereby obtaining a fluid-dependent release of the active substance. In this example, the active substance is released substantially independently of fluid which is present in the continuous phase within the bandages. The prior art also discloses cutting sheets of bandages having biologically active material incorporated therein into corpuscles that are placed on the patient's treatment site. An astringent haemostatic preparation provided with a granulated haemostatic (an inorganic aluminum or ferric salts) is also known in the prior art.

The treatment of chronic wounds, ulcers and the like has severe limitations when topical application of anti-microbial agents are administered. Anti-microbial agents are generally ineffective in wound healing largely due to leaching of the anti-microbial agent from the wound site and the inability to be able to maintain an effective amount of the active agent in contact with the wound site. An ideal wound dressing should remove excess exudates from the wound while keeping the wound moist, prevent dehydration (wound exudates are a bactericide which if left in position in moderate amounts tends to speed up the healing process), allow gaseous exchange, provide thermal insulation, be impermeable to micro-organisms, have low adherence properties, and be free from particulate and toxic contaminants.

In one exemplary embodiment, the method and compositions of the present invention incorporate a gauze pad in an elastic bandage which is adapted to conform to the treatment site of a subject. The textile material of the gauze pad incorporates the anti-microbial composition of the present invention within the interstitial spaces of the polymeric fiber material from which the gauze pad is fabricated.

Advancement in modern fiber technology has enabled low concentrations of biocides to be incorporated into the fibers of the bandage or dressing. This prevents broad-spectrum microbial growth in the target zone and allows the agents to remain effective over longer periods. However, there is room for improvement with this type of product especially in the area of more efficient delivery of therapeutic agents to the wound care site with increased therapeutically effective concentrations.

Accordingly, there is a need for methods and compositions which can prevent broad spectrum microbial growth, reduce inflammation in the targeted zone, and also encourage wound healing.

### SUMMARY OF THE INVENTION

One object of the present invention is to provide a therapeutic composition which suppresses infectious microorganisms (i.e. bacteria, viruses) and infection by promoting the electrical current density to wounds.

It is another object of the invention to provide a composition incorporated into a wound care system which is self-adhering to a wound yet easily releasable when use is discontinued.

It is yet another object of the invention to provide a wound care system which draws wound seepage away from the damaged or infected skin of a user to reduce the opportunity for infection and to enable the system to be left in place for extended periods of use.

It is still another object of the present invention to provide new or improved wound care dressings or bandages comprising therapeutically effective amounts of a therapeutic agent of the inventive composition to overcome or at least minimize the prior art problems or limitations outlined above.

It is another object of the present invention to provide an improved therapeutically active agent or combination of agents useful in wound care management for the promotion of wound healing.

It is yet another object of the present invention to provide new compositions that substantially improve a patient's localized electrochemical environment, and which alleviate the inflammatory process thereby promoting improved wound care management and wound healing, and decreasing the associated pain.

This invention encompasses an expeditious and long-lasting method and compositions for treating a wide variety of wounds and injuries by promoting the elevation of pH at the site or sites of injury, enhancing the reduction or elimination of the patho-physiological process of infection proliferation, and promoting better wound care management and wound healing. The invention also provides a method of treating a subject having a condition associated with the presence of free radicals in quantities sufficient to cause undesirable symptoms.

The present invention encompasses controlling mechanisms of intracellular and extra-cellular ionic physiology through the administration of alkaline salts for pH modulating and for restoring and enhancing the localized and/or systemic cellular ionic physiology. The composition promotes the increase of the electrical current density to certain types of wounds and simultaneously normalizes the localized ionic environment, thereby normalizing immune responses and reducing acidotic induced pain and pathogen induced pain.

The biological environment of pathogens and anaerobic cells have a narrow and specific viability zone and oxidation reduction potential (ORP) range limited to a narrow pH range, generally ranging between about 5.20 to 6.80, while infectious microorganisms have viability zone ranges between about 5.00 to 6.80. Healthy human cells function in a pH range and ORP outside of the pathogen's viability zone. Administering the composition of the present invention increases negative hydrogen ions thereby increasing the pH in the fluids and cells. As a result, the electrons tend to move fluids toward an ORP and pH that are in a range consistent with optimum physiologic aerobic metabolic functions and the optimum metabolic function and systemic pH promote cellular function and repair.

One exemplary embodiment of the present invention includes a method for the administration of the composition of the present invention with electrolytes (saline) thereby enhancing the ability of immune cells, including macrophages, natural killer cells and T-cells, to promote functional equilibrium. This results in an enhanced immune surveillance. It is known that more acidic conditions (reduced pH) create a status of chronic immune stimulation and inflammation which is evidenced by impaired functional immune balances. Restoration of the proper physiological (mild alkaline) pH strongly improves immune function and the interaction between the immune system and the psychoneuroendocrine system (e.g. hypothalamus-pituitary-adrenal axis).

The present invention encompasses a method and compositions for wound care that i) suppress a wide variety of infections that occur during surgery, ii) shorten recovery periods by promoting the healing cycle of normal, healthy, viable cells, and iii) minimize scarring commonly associated with surgical procedures or burns. In addition, residual damaged or infected cells encounter a more hostile environment (more alkaline pHe) and, as a consequence, are rendered nonviable and are eliminated.

The inventive methods and composition of the present invention include using the composition in the form of additives to soaps, deodorants and toothpastes. The present invention also includes incorporating the composition in textile materials and yarns. In addition, the composition of the present invention may be incorporated into clothing to control the growth of microorganisms between launderings. Other common applications may include, but are not limited to, incorporation in animal beds, dental floss, shoe innersoles, furniture coverings and public transport seating. In the medical field, the composition may be incorporated into the material used for hospital bed sheets, surgical drapes, hospital gowns, operating gowns, and medical masks. Additional medical applications include incorporating the composition of the present invention in bandages, gauze, filters, and anywhere a textile or textile fiber could be used to control mold, mildew, fungus, yeast or bacterial growth.

The present invention also provides a method and composition for treating a wound, a burn, or any other condition associated with the presence of free radicals in quantities sufficient to cause undesirable symptoms. The compositions of the present invention include an effective amount of cesium and/or rubidium ions and a pharmaceutically acceptable carrier and/or one or more surfactants. The composition may also include an antimicrobial composition that includes a suitable carrier and cesium and/or rubidium ions in an amount effective to suppress the growth of a wide variety of infectious microorganisms or opportunistic pathogens.

The composition of present invention may be incorporated into orthotic devices and compression garments, such as ACE™ bandages, which can be applied to curved skin surfaces as needed. The invention also relates to corpuscles that can be adhesively placed on the side of a dressing intended to face the skin of the patient. The corpuscles consist of biologically active compositions in wound care and are further characterized in that the biologically active substance or substances is/are dissolved or dispersed in at least one hydrophilic phase discontinuously present in the matrix of the dressing or present as a coating thereon.

A large number of deaths are due to secondary infections, usually from bacteria invasion. If a patient's immune system is suppressed by bacterial-induced acidosis, then the inventive composition described herein will contribute to the enhancement of the functioning of the localized and systemic immune system thereby promoting resistance to secondary infections. The composition of the present invention suppresses a wide variety of infectious microorganisms, i.e. bacteria, which often have acidotic energy metabolisms. Antibodies and phagocytes cannot work effectively in tissue having increased acidity.

In another exemplary embodiment of the present invention, the method and composition of the present invention act as an adjunct simultaneously or sequentially with surgical procedures for reducing scar tissue formation from surgical procedures. Scar tissue occurs when there is an incorporation of damaged or dysfunctional cells in the tissues, particularly when the immune system is enveloped in the healing tissues from infected cells at the site of damage. Normal healthy viable human cells optimally function in a pHe range between about 7.31 to 7.45 with a pHi ranging between about 6.60 to 6.80. The cesium and/or rubidium ions in the composition of the present invention promote pHe and pHi elevation which facilitate the destruction of damaged or defective cells thus promoting less scarring during the healing process. The ions also simultaneously enhance the tissues' healing process at the site of the damage.

The method and composition of the present invention may also be used to treat and/or prevent adhesions that occur as a result of injuries and/or surgery. Inflammation, surgery, or injury can cause tissues to bond to other tissues or organs thereby forming adhesions. Adhesions can cause a number of disorders and complications, many of which may be life-threatening. The composition of the present invention may be administered in the form of a topical wash or rinse to treat and/or prevent adhesions. Adhesions may also be treated by directly injecting the composition of the present invention.

Yet another exemplary embodiment of the present invention includes administering the composition in the form of a bandage or gauze for wound treatments. For example, the composition may be incorporated into a bandage and then topically administered with a pH range between about 6.00 to 9.5, more preferably between about 7.00 to 7.50 with an Oxidation Reduction Potential (ORP) ranging between about −1 mV to −100 mV, more preferably ranging between about −1 mV to −25 mV.

Administering a sufficient dose to the injured sites and cells establishes an electro-minus charge which promotes the electrical flow while simultaneously reducing scarring and pain. The present invention promotes the increase of the electrical current density to certain types of wounds which increases blood flow thereby increasing the healing process. As a result, a wide variety of pathogens are killed at an increased rate thus minimizing the likelihood of prolonged infection, scarring, tissue swelling, and pain.

The composition of the present invention encompasses a mixture of cesium and/or rubidium salts and, optionally, minerals such as electrolyte concentrations which mix with body fluid to form an isotonic state. The composition typically comprises halide salts of sodium, potassium, calcium, and other cations with preference given to halide salts of chloride, sodium, potassium, and magnesium. A ratio of sulfate and citrate is the most preferred form. The preferred ratio of cesium sulfate to cesium citrate is about 3 cesium sulfate to about 2 cesium citrate. An optimum ratio of cesium sulfate to cesium citrate includes a ratio of between about 2 to 3 cesium sulfate to about 1 cesium citrate. It is preferred that the cesium sulfate proportion of the ratio should always exceed that of the cesium citrate. The combination of cesium sulfate and cesium citrate preferably comprises a range of about 0.10% to about 5% of the composition.

The composition of the invention is atoxic and has antibacterial properties. The composition is useful in any application in which antimicrobial properties are desirable, particularly in the topical application to wounds, burns, etc., and can be incorporated into a bandage or other type of wound dressing. A combination of cesium salts are preferred

The composition of the present invention may comprise a physiologically balanced, ionized, salt (saline) solution containing cesium and/or rubidium ions. The composition of the invention is prepared using a combination or mixture of salts in physiologically balanced proportions. A mixture of inorganic salts and, optionally, minerals (such as metallic elements, for example, and not by way of limitation) is used in order to complement the electrolyte concentration and mixture of body fluid in an isotonic state. The solution typically comprises salts of sodium, potassium, and other cations and preferably includes cesium salts which include, but are not limited to, citrate, malate, sulfate, carbonate, iodide, and chloride. However, cesium chloride should be used with caution.

The solution may optionally contain other salts or minerals. The pH range of the solution is about 6.00 to 9.50, more preferably ranging from about 7.00 to about 7.70, with an ORP (Oxidation Reduction Potential) within the range of about −10 mV to about −100 mV, and more preferably within a range of about −10 mV to about −50 mV.

For anti-bactericidal, fungicidal, and sporicidal properties, the composition's pH is about 7.50 or above up to about 9.50. The composition is physiologically balanced by the inclusion of elements such as sodium, potassium, magnesium, zinc, manganese and magnesium. Typically, these elements are supplied in the form of salts that are readily ionized. Preferably, these physiologically balancing salts are selected from the group consisting of cesium salts and combinations thereof. The salts are preferably selected from cesium sulfate and cesium citrate.

The compositions of the present invention are physiologically balanced and, when applied to infected wounds, substantially enhance the process of healing and wound closure. Antimicrobial properties of the composition may reduce or eliminate the following: Escherichia coli, Listeria monocytogenes, Staphylococcus aureus, methicillin-resistant S. aureus (MRSA), Pseudomonas aeruginosa, Lactobacillus, yeast, vancomycin-resistant enterococcus and other vancomycin-resistant bacteria, molds, and spores. The compositions of the present invention are osmotically balanced, environmentally friendly, and have minimal cytotoxicity.

The compositions of the present invention are nontoxic and have antibacterial properties. They are useful in any application in which antimicrobial properties are desirable. Such applications include, without limitation, treatment of wounds, burns, and canker sores; irrigation; cleaning of tissue sites (e.g., pre- and post-operative); dermatological applications, treatment of psoriasis; and numerous applications which are readily apparent to one skilled in the art. Unlike many other compositions used in similar applications, the composition of the present invention has minimal to no side effects.

Examples of the inventive composition may contain biologically active substances which may suitably be applied to wounds, including abrasions and burns, by means of a dressing material. The inventive composition may also include haemostatic and antiseptics such as iodine and iodine compounds (e.g. a complex of iodine and a 1-vinyl-2-pyrrolidone-homopolymer for the treatment of burns), silver nitrate, silver acetate, chlorohexidine, benzalkone and chloramine; antibiotics such as neomycine, amphotericine, fusidic acid and the tetracyclines; anti-inflammatory agents, e.g. anti-inflammatory steroids such as cortisone, hydrocortisone, betametasone and derivatives thereof, prednisone and alkylderivatives thereof, prednisolone, triamcinolone and derivatives thereof such as the acetonide, and dexametasone, or non-steroid anti-inflammatory substances such as salicylic acid and derivatives thereof, especially acetylsalicylic acid; local anesthetics such as lidocaine, bupivicaine, tetracaine, cincocaine and benzocaine salol; anabolic steroids for building up tissues under wound healing, e.g. metandienone; proteolytic agents for the decomposition of fibrin, e.g. trypsine; vasodilating substances for improving the flow of blood during wound healing, e.g. tolazoline; and heparin and other thrombosis-hampering substances.

Another exemplary embodiment of the present invention includes a wound care kit which includes a package containing a composition of the present invention and a bandage or other type of wound dressing.

### DETAILED DESCRIPTION OF THE INVENTION

Definitions

As used herein viscosity is measured in dynes per cm² at 20 degrees C.

As used herein acidity and alkalinity are measured by pH which is defined as the negative logarithm of the hydrogen ion activity: pH=−log(H). The parameter pHe is the pH on the exterior of the cell and pHi is the pH on the interior of the cell.

The term “reducing acidity” or “to reduce the acidity of” as used herein in relation to the tissues of a mammal means to raise and/or maintain the pH, pHe and pHi such the various pH's are above the action potentials responsible for nerve conduction (i.e. a pH of 6.80 or higher).

As used herein, “about” is defined as plus or minus 2.5%, unless specifically stated otherwise.

As used herein, “pharmaceutically acceptable carrier” includes any and all solvents, surfactant(s), dispersion media, coatings, antibacterial and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions is contemplated.

As used herein, “cesium sulfate” also means dicesium sulfate, sulfuric acid, and discesium salt.

As used herein, “cesium citrate” also means 2-hydroxy-1,2,3-propanetricarboxylic acid salt.

As used herein, “glycerin” also means glycerol, glycerine, propane-1,2,3-triol, 1,2,3-propanetriol, 1,2,3-trihydroxypropoane, glyceritol, and glycol alcohol. Glycerin a sugar alcohol that has three hydrophilic alcoholic hydroxyl groups (OH) that are responsible for its solubility in water.

As used herein, “DMSO” also means dimethyl sulfoxide, methyl sulfoxide, and methylsulfinylmethane. The molecular formula for DMSO is C₂H₆OS. Related compounds may also be used which include diethyl sulfoxide, dimethyl sulfoxide, and dimethyl sulfone.

For the purposes of this specification it will be clearly understood that the word “comprising” means “including but not limited to”, and that the word “comprises” has a corresponding meaning.

Method of Manufacture

Principal Active Ingredients

This invention incorporates salts containing cesium ions and/or rubidium ions, as a stand-alone wound care therapy, or as an adjunct in conjunction with a wide variety of conventional therapies. The compounds of the present invention may be crystallized with counter-anions. All previously described compounds and complexes can be crystallized with other counter-anions. Those anions that give the best activity and lowest toxicity were chosen here.

The cesium salts included in the composition of the present invention may be formed using a variety of acids, including, but not limited to: carbonate, chloride, citrate, lactate, nitrate, phosphate, and sulfate. Carbonate, citrate and sulfate salts are the safest and phosphate salt is relatively safe but may interact with calcium. Chloride salt should be used with caution. A ratio of citrate and sulfate is the most preferred form. The preferred ratio of cesium sulfate to cesium citrate is about 3 cesium sulfate to about 2 cesium citrate. An optimum ratio of cesium sulfate to cesium citrate includes a ratio of between about 2 to 3 cesium sulfate to about 1 cesium citrate. The cesium sulfate proportion of the ratio should always exceed the cesium citrate. The combination of cesium sulfate and cesium citrate preferably comprises a range of about 0.10% to about 5% of the composition. Cesium citrate alone is also effective for wound therapy.

Additionally, other cesium and rubidium salts may be used in a variety of compositions if they meet the following requirements: (1) they are pharmaceutically acceptable and have an acceptably low level of toxicity; and (2) they have sufficiently high levels of cationic dissociation to allow the remaining negatively charged ions to effectively reduce acidity.

Surfactants are used in the composition of the present invention as penetrating agents, dispersing agents, solubilizing agents and spreading agents. Some examples of surfactants are: PEG (polyethylene glycol) 400; Sodium lauryl sulfate; sorbita palitate, sorbitan laurate, sorbitan stearate available under the tradename Spans® (20-40-60 etc.); polyoxyethylene 20 sorbitan monolaurate, polyoxyethylene (20) sorbitan monopalmitate, polyoxyethylene (20) sorbitan monostearate tradename Tweens® (polysorbates, 20-40-60 etc); and Benzalkonium chloride. Examples of other suitable surfactants may include alkyl sulfates; condensation products of ethylene oxide with fatty acids, fatty alcohols, fatty amides, polyhydric alcohols (e.g. sorbitan monostearate, sorbitan oleate), alkyl phenols (e.g. Tergitol) and polypropylene oxide or polyoxybutylene (e.g. Pluronics); amine oxides such as dimethyl cocamine oxide, dimethyl lauryl amine oxide and cocoalkyldimethyl amine oxide (Aromox); polysorbates such as Tween 40 and Tween 80 (Hercules); sorbitan stearates, sorbitan mono-oleate, etc.; sarcosinates such as sodium cocoylsarcosinate, sodium lauroyl sarcosinate (Hamposyl-95 ex W.R. Grace); cationic surfactants such as cetyl pyridinium chloride, cetyl trimethyl ammonium bromide, di-isobutyl phenoxy ethoxy ethyldimethyl benzyl ammonium chloride and coconut alkyl trimethyl ammonium nitrate. Glycerin and D.M.S.O. are the preferred surfactants.

Suitable surfactants are those that are low toxicity reasonably stable throughout a wide pH range, including non-sop anionic, nonionic, cationic, zwitterotic and amphoteric organic synthetic detergents. The purpose of using surfactants in the preferred compositions of the present invention is to adjust the surface tension of the composition as a penetrating agent and/or to improve site direction so that the maximum amount of the composition is deposited in or near the nucleus of the infected cell sites. Surfactants may be used to adjust the viscosity to the therapeutic ranges as stated herein from about 8 dynes/cm² to about 36 dynes per cm², more preferably ranging between about 10 to about 30 dynes/cm² to effectively penetrate the necrotic cells and tissues in a wound.

Carriers

One exemplary embodiment of the present invention includes active compounds prepared with surfactant(s) and/or carriers that protect the compound against rapid elimination from the body such as a controlled release formulations, including implants and microencapsulated delivery systems. Biodegradable or biocompatible polymers can be used such as ethylene vinyl acetate, polyanhydrides, polyglycolic acid, collagen, polyorthoesters, and polylactic acid. Such materials can be obtained commercially from ALZA Corporation (Mountain View, Calif.), NOVA Pharmaceuticals, Inc. (Lake Elsinore, Calif.), or prepared by one of skill in the art.

Potentiation of cesium and/or rubidium ionic action can be accomplished by inclusion of ingredients that enhance ionic pH physiology. Examples include electrolytes (saline compounds) such as potassium, sodium, and magnesium, and other major electrolytes such as calcium, chloride, bicarbonate, phosphate, and sulfates. Other ingredients that may be included to effectively potentiate the wound site-specific affinity of cesium/rubidium ionic action include manganese, vitamin B6 (pyridoxine), Vitamin D2, Vitamin D3, a mixture of Vitamin D2 and D3, and/or Vitamin B12.

Modes of Manufacture

After determining the dose to be used in the composition, each ingredient is weighed/measured out individually, added together and dissolved in sterile composition. The preparation is then tested to ensure that it is within the parameters established for surface tension, viscosity, osmolarity, ORP, pH, and sodium chloride equivalency. This is done by using the appropriate equipment for each test.

In one exemplary embodiment, a combination of cesium salts are weighed/measured out individually, added together, and dissolved in sterile glycerin and D.M.S.O. for administration. The preparation is then tested to ensure that it is within the parameters established for surface tension, osmolarity, pH, and sodium chloride equivalency. This is done by using the appropriate equipment for each test. To prepare a unit dose, the ingredients of such formulations generally will be dissolved in a carrier and surfactant such as glycerin and further lowering the viscosity with DMSO or other suitable surfactant(s).

The present invention provides a composition(s) and method for treating a subject having a condition associated with the presence of free radicals in quantities sufficient to cause undesirable symptoms. The composition comprises a pharmaceutically acceptable carrier and cesium and/or rubidium ions or compounds in an amount effective to alleviate the undesirable symptoms associated with the presence of the free radicals. A “pharmaceutically acceptable carrier” includes any and all solvents, surfactants, dispersion media, coatings, antibacterial and antifungal agents, isotonic and absorption delaying agents, and the like, compatible with pharmaceutical administration (Remington 2000). Preferred examples of such carriers or diluents include water, saline, Ringer's solutions and dextrose solution. Supplementary active compounds can also be incorporated into the compositions. The method of the present invention comprises administering the composition to the subject in an amount effective to alleviate the undesirable symptoms.

The method may be used for the treatment of any condition associated with free radicals. However, it is most effective against conditions associated with oxygen free radicals. Such conditions may comprise inflammation, including synovial inflammation and arthritis, ulcers such as diabetic ulcers, and conditions caused by the loss of circulation due to free radicals, such as hair loss due to the loss of dermal microcirculation.

Methods for Using the Composition of the Invention

The cesium and/or rubidium salt compositions of the present invention enhance the healing process of any wound contaminated with microorganisms. The compositions of the present invention function specifically to maintain the necessary antibacterial environment for wounds to heal faster, without the usual complications associated with superficial infections. In addition, the compositions provide topical bacterial control with less pain, faster wound closure, and reduced scarring.

The solution may be applied with the help of a cotton swab for more specific areas. The solution may also be used once or several times a day according to the individual patient's needs and condition. The solution may be applied by soaking a gauze bandage material with the solution and applying the saturated gauze to the affected area.

One skilled in the art will see that the composition(s) of the invention have applications in the treatment of many different types of wounds, including, without limitation, diabetic ulcers, gangrene, venous ulcers, decubitus ulcers, pressure ulcers, wounds due to bites, acute trauma wounds, surgical wounds, and burns. The composition of the invention can also be used for pre- and post-operative cleaning of tissue sites, and as a gargling solution for treatment of canker sores etc. In addition, because of its electron donors, the solution of the invention may be a strong growth factor stimulator in the wound healing process. As such, the solution may find uses in many other applications in which disinfection and growth factor stimulation are desirable.

Method of Wound Care

Patients in the clinical environment suffering from long-lasting non-healing wounds may be treated with the topical composition of the present invention, typically ranging between 1 to 2 times per day, generally about once per day or as necessary depending on the severity of the wound. The composition of the invention may be used in place of a saline solution to control infection and to help the wound healing mechanisms. The solution of the invention may be used by presoaking a gauze material or gauze pad with sufficient composition. This process removes species present in the gauze that would react with, and reduce the effectiveness of, the solution of the present invention. The gauze should be moist with the composition but not completely soaked. Additional solution is then applied to completely saturate the gauze, which is then immediately applied to the wound. In the alternative, the composition may also be used to clean a wound by pouring or spraying it directly on the wound site to remove any necrotic tissue by a mechanical procedure, and also as a cleanser or irrigant. The gauze may be applied to the wound and, if necessary, additional solution applied. Typically the wound site is packed with the solution-soaked gauze, or as a variation, a suitable gauze can be applied on top of the packed wound to keep it moist and free of contaminating germs. The wound site is then wrapped with wound dressings as is standard in the art.

According to one exemplary aspect of the present invention there is provided a bandage or dressing for wound care management which includes an outer fabric support, preferably an elastomeric fabric support, and an inner pad, wherein the inner pad includes an outer membrane surface, preferably fabricated into a film-forming material incorporating a therapeutically effective amount of one or more therapeutically active (e.g. anti-microbial) compounds including salts of cesium and/or rubidium in the matrix thereof. The therapeutically active compounds preferably comprise a combination of cesium sulfate and cesium citrate. The pad may be integral with, or separate from, the outer fabric support. The inventive composition is preferably incorporated into the membrane matrix, but may also be incorporated into the material of the inner pad contained by the membrane. The therapeutically active agent or agents are held in the polymeric matrices so that migration is inhibited, thereby causing the controlled release of the cesium agents. According to the present invention, the term “therapeutically effective amount” means an amount of therapeutic (anti-microbial) agent and/or mixture thereof which is capable of promoting wound healing and retarding or preventing microbial colonization and adherence to the surface of the materials used herein while in contact with living tissue causing minimum undesirable side effects.

As previously described, the composition of the present invention can be incorporated into a bandage or wound dressing. The use of more than one active disperse phase may be applied to adjust the velocity at which the active substance or substances are released to the wound. Such adjustment may take place in a variety ways. One way is to have discontinuous phases which are hydrophilic in different degrees whereby they are dissolved at an uneven rate in the wound exudate. Another way is to have the concentration of identical biologically active substances be different in different hydrophilic phases that are active in wound care, including those systems in which the biologically active substance is protected against bacterial decompositions in the clinical situation. Many biologically active substances usable in wound care, e.g. enzymes, are sensitive to being decomposed by compounds such as proteases which are frequently present in wound secretions. Such decomposition may be counteracted by coating the phase or phases with an antiseptic, or by the presence of the antiseptic and the enzyme in separate phases. A third way of adjustment is to have different phases active in wound care contain different biologically active substances having fundamentally similar or fundamentally dissimilar biological activities. Optionally, one phase that is biologically active in wound care may contain a haemostatic—e.g. an inorganic aluminum salt or a ferric salt—in a parent material that is readily soluble. As a result, a rapid onset of a haemostatic effect is attained while one or more other substances active in wound care will be effective over a longer period of time.

Another exemplary embodiment of the present invention encompasses a method and composition which includes a hydrophilic material or a hydrophobic material which is immiscible with the matrix and which has a content that generates gaseous substances under the influence of the wound exudates. The material may be present as one or more biologically passive discontinuous phases. The content may include a solid acid such as, for instance, lactic acid or citric acid and a salt of a carbonic acid that by contact with the acid will form carbon dioxide. This phase may function to establish a foam-like structure in the matrix in order to promote a comparatively rapid release of the substance that is biologically active in wound care. Gases such as carbon dioxide formed by bicarbonate and citric acid may also be effective in the displacement of oxygen. Certain types of wounds must be exempted from free oxygen in that free oxygen may hamper the angionesis.

### WOUND CARE EXAMPLE 1

A formula containing cesium chloride and olive oil was topically administered for 3 consecutive days to a 42-year-old male having a slow-healing painful wound on his shin approximately 5 to 6 cm in diameter. The wound healed in about 4 days, and the underlying skin looked pink and younger than the surrounding skin.

### WOUND CARE EXAMPLE 2

A formula containing cesium chloride and olive oil was topically administered to a 40-year-old male having a slow-healing painful wound with a nodule on his small finger. The wound healed in about 3 days, and the nodule disappeared.

Description of the Wound Care Kit

The patient may also make use of a “wound care kit” which permits the patient to periodically pour the solution of the present invention onto the wound site without having to remove the dressing. The kit provides ease-of-use, portability and dramatically reduces exposure of the wound. The wound care kit includes a package containing the composition of the invention and bandaging material. The composition can be poured directly through the bandage without changing the dressing, thereby reducing the number of dressings, associated time, risk and trauma. The bandage helps keep the skin surrounding the wound dry while the wound is treated. The bandage and solution in the wound care kit may be applied in a physician's office or at a hospital with the patient continuing care at home. The wound care kit may also be applied and used at home under the instructions of a physician or, for minor injuries, the wound care kit may be used as an “over the counter” treatment by the patient alone.

The wound care kit includes bandaging material and a package of the solution of the present invention. Preferably, the packaging material provides a non-reactive surface with the solution/composition. In addition, the bandaging material preferably includes a specially designed wound “bandage” made out of an oxygen-permeable bandage material to prevent the wounded tissue from drying. The bandage is described in more detail herein. The kit may also include gauze or a similar material for packing of the wound, to be used in combination with the solution and a bandage. Instructions are optionally included for administration of the composition by a physician or over the counter by the patient.

Another exemplary embodiment of the present invention encompasses a complex compound or a combination of complex compounds that can be administered subcutaneously or topically in a suitable vehicle, e.g. physiological saline in the case of subcutaneous administration, and glycerin and/or dimethylsulfoxide (DMSO) in the case of a topical administration, although ointments, salves or like conventional vehicles may be employed. The dose may be administered one to three times daily, depending upon the severity of the inflammatory condition, preferably under medical supervision so that the dosage can be reduced or the number of daily administrations limited as the inflammatory condition subsides.

Yet another exemplary embodiment of the present invention encompasses an antimicrobial composition that comprises a suitable carrier and/or surfactant and cesium and/or rubidium ions in an amount effective to suppress the growth of microorganisms. The antimicrobial composition or compounds of the present invention may be used for the treatment of a wound or burn by topically administering to the wound or burn the inventive composition or compounds. The compounds may also be used in the treatment of conditions that are normally treated with antimicrobial or antibacterial agents. For example, the compounds may be used in the topical treatment of an infectious disease or an abrasion as an antibiotic.

The compositions of the present invention are water-soluble and may be dissolved in a number of carriers. Suitable carriers may include a surfactant or surfactants, polar solvents, protic solvents such as water, or especially normal saline.

The composition may be applied to the site of the burn, wound, abrasion, etc. in the form of a wet or dry aerosol, in the form of a salve, ointment, or cream, or directly in the form of a liquid solvent, preferably normal saline, by the use of a medicine dropper. Furthermore, the composition or complex compounds may be applied to the burn site together/simultaneously or sequentially with a topical anesthetic agent such as benzocaine, a soothing agent such as menthol and/or eucalyptus, an antibacterial agent such as bacitracin, or a combination of these ingredients as necessary.

The inventive composition is particularly effective for killing microorganisms such as, but not limited to, Strap. B hemolytic, Strap. alpha. hemolytic, Enterococci, Staph. coagulase (+), Staph. coagulase (−), E. Coli, Klebsiella, Pseudomonas, Proteus, and C. albicans. It is also contemplated that the compounds and compositions of the present invention may be used in the treatment of other conditions associated with free radicals, such as poisonings with pharmacologic agents or conditions caused by ionizing radiation, etc.

The composition of the present invention is formulated to be compatible with its intended route of administration, including subcutaneous, oral, transdermal, transmucosal, and rectal administration. Solutions and suspensions used for parenteral, intradermal or subcutaneous application may include a sterile diluent, such as water for injection, saline solution, D.M.S.O., polyethylene glycols, glycerin, propylene glycol or other synthetic solvents; antibacterial agents such as benzyl alcohol; antioxidants such as ascorbic acid, sodium bisulfite, or sodium bisulfate; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Administration can be transmucosal or transdermal. For transmucosal or transdermal administration, penetrating agents that can permeate the target barrier(s) are selected. Transmucosal penetrating agents include surfactants, detergents, bile salts, and fusidic acid derivatives. Nasal sprays or suppositories can also be used for transmucosal administration. For transdermal administration, the active compounds are formulated into ointments, salves, gels, or creams. Suppositories (e.g., with bases such as cocoa butter and other glycerides) or retention enemas for rectal delivery may also be prepared.

Accelerates Wound Healing

This present invention relates to a topical composition comprising cesium and or rubidium salts particularly, but not exclusively, for impregnating a bandage with pastes or gels containing the composition.

Another exemplary embodiment of the present invention encompasses a method for providing a topical composition for impregnating a bandage comprising at least one viscosity-reducing surfactant and a carrier, preferably in the absence of a preservative.

Yet another exemplary embodiment of the present invention encompasses compositions that are primarily intended for impregnating bandages for use in the treatment of a wide variety of skin diseases.

Still another exemplary embodiment of the present invention encompasses surgical rinse solutions that are used either during or immediately following surgery to irrigate body tissues. These rinses contain a solution of cesium and/or rubidium salts and reduce the incidence of edema and adhesions resulting from surgery.

Another exemplary embodiment of the present invention encompasses a composition suitable for use in medicine, in particular as an infusion or surgical rinse solution, comprising a carrier solution.

Preferred forms of the compositions of the invention contain an effective concentration of cesium and or rubidium salts, e.g. a bacterially effective concentration or an anti-toxin/anti-bacterial concentration. Such compositions may be used in the form of a solution in the treatment of infection or sepsis or as an anti-mediator therapy.

One key to topical administration is delivering the composition in a low or suitable viscosity range. For example, a composition having a viscosity below 36 dynes per cm² will seep in between the cells, may also be transferred osmotically from cell to cell, and is capable of going into the subcutaneous tissues. The preferred viscosity of the composition is below 36 dynes per cm².

In one exemplary embodiment, the composition of the present invention may contain a concentration of about 1/10 of one percent combined cesium sulfate and cesium citrate which is preferably adjusted to a neutral pH with a viscosity range from about 15 to 25 dynes/per cm².

The composition of the present invention preferably avoids surfactants that contain toxic substances such as petroleum based chemicals that may be transported into healthy cells or that may produce other toxic compounds.

The present invention encompasses administering the inventive composition topically during surgery such as a wash or a rinse. The composition suppresses bacteria and the secondary effect of bacteriological invasion.

The composition of the present invention is particularly useful in topical applications. The composition may be incorporated into bandages as an anti-bacteriant. The composition also reduces scar formation and promotes the healing of the affected tissue from the inside of the tissue. The composition functions to osmotically shield healthy cells and disassociate the damaged unshielded cells which are then metabolized thereby reducing scar tissue and adhesion formation and infection.

The present invention encompasses administering the composition of the present invention in the form of a bandage or gauze for wound treatment. As an example, the composition can be topically administered with a pH ranging between about 6.00 to 9.50 more preferably between about 7.00 to 7.50, and an ORP ranging between about −1 mV to −50 mV, more preferably from about −10 to −25 mV by administering a suitable dose to effect the injured sites thereby establishing an electro-minus charge to promote the electrical current flow that simultaneously reduces scarring and pain. Administration may be intravenous, intra-peritoneal, parenteral, atomized, intramuscular, subcutaneous, oral, or topical, with topical being more preferred.

The cesium and or rubidium in the composition of the present invention have an important added advantage due to the inherent bio-localization, site-directing, or affinity to damaged or necrotized cells and tissues. “Inherent bio-localization” for targeted in vivo and vitro delivery means having specificity for targeted sites for damaged tissues.

In another exemplary embodiment of the present invention, the method and composition encompasses the administration of the composition with electrolytes (saline) and supporting nutrients to enhance the ability of immune cells including macrophages, natural killer cells, and T-cells, to promote functional equilibrium thereby enhancing an improved immune surveillance for necrotized cells. It is known that more acidotic conditions (reduced pH) create a status of chronic immune stimulation and inflammation which is evidenced by impaired functional immune balances. Restoration of the proper physiological (mild alkaline) pH strongly improves immune function and positively influences the interaction between the immune system and the psychoneuroendocrine system (e.g. hypothalamus-pituitary-adrenal axis). Thus, the inventive composition has localized immunomodulatory activity.

The present invention encompasses administering sufficient quantities of the composition to enable normal healthy viable cells and tissues to act as a barrier to sufficiently electro-physically block infection from pathogens, i.e. bacterial invasion. The composition elevates and enhances the ability of normal, healthy cells to electro-physically resist the decreased pHe and pHi that occurs in injuries and wounds, and promotes rapid healing.

Administration of sufficient quantities of the composition in an amount to effectively treat a patient's wounds elevates the localized pH, enhances immune response for pathogen suppression by increasing the ionic concentration in the adjacent viable healthy normal cells, and elevates the pHe and pHi to a physiological more optimum range that reduces the injury site's acidotic pH and pHi. This simultaneously enhances the resistance against acid-mediated invasion of a wide variety of bacteria and other opportunistic pathogens.

The present invention also encompasses administering a composition for suppression and elimination of acidotic infection-generated induced pain. Sufficient quantities of the inventive composition are administered to modulate the patient's pHe to a sufficient level above 6.80, preferably above 7.21. As a result, the patient's electro-physical function is restored including physiologically improving intracellular energy production and normalizing metabolism. The compositions of the present invention may be used to treat the pain and discomfort associated with this condition. Cesium ions in the composition of the present invention will eliminate pain and are preferably included in an amount of about 2,000 ppm to about 100,000 ppm.

The inventive compositions of the present invention may also be administered in conjunction, sequentially or separately, with other topical compositions. The compositions are typically applied in the form of a liquid, a paste, a gel, an impregnated fabric strip (e.g., bandage) or an impregnated surgical sponge.

Compositions of the present invention incorporated into bandages preferably include cesium and/or rubidium salts, preferably comprising a combination of cesium sulfate, cesium citrate, cesium carbonate, cesium nitrate and cesium chloride combined with Vitamin B6 (pyridoxine) and or Vitamin B12. Cesium sulfate and cesium citrate are most preferred.

If a patient's immune system is suppressed by an acidic oral biological environment (whether induced by radiotherapy or chemotherapy, viral or bacterial-induced, or age-related), the composition and therapy described herein provides for an increase (elevation) of the pH and ORP to more optimum ranges during the therapy. This stimulates the patient's immune response and functions to resist a wide variety of infectious organisms and diseases. The composition is effective in minimizing levels of pain that occur in a reduced pH region, particularly in the oral region of the mouth and throat.

The present invention discloses a method and composition that elevates the pH and the electrophysiological environment from one that promotes a wide variety of acidotic induced inflammation and pain to a pH range that potentiates a more optimum immune function. Increasing the pH promotes the elimination of the pathogen's viability zone (electrophysiological environment), promotes regeneration of cells and tissues (wound healing) by inducing a more optimum electro-physical cellular function, reverses molecular pathology, and restores cellular electrochemical equilibrium. Secondarily, by stabilizing or eliminating the acidosis state and increasing oxygenation in the localized electro-biological environment, the hostile effects caused by acidosis (reduced pH) are minimized and eliminated, and more optimum pH ranges are restored.

The composition and method will eliminate the acidosis state so that the physiologic cellular pHe approaches optimal, or near optimal, ranges between about 7.21 to about 7.55, preferably between about 7.37 to about 7.41. If pHe is close to optimum physiologic levels, metabolic function is not compromised and cellular regeneration and repair takes place.

One aspect of the present invention encompasses a composition for use as a rinse. The composition includes at least one cesium salt and aqueous solution having a surface tension ranging from about 10 to about 36 dynes per cm², more preferably ranging between about 10 to about 30 dynes per cm².

The present invention also encompasses a method for reducing inflammation. The method involves applying a composition comprising from about 1,500 ppm to about 100,000 ppm, and preferably about 2,000 ppm to about 50,000 ppm, of cesium and/or rubidium ions to the inflamed area. Cesium is preferred. Without available electron donors at the site of an injury, the pH of the electro-physical environment is reduced and compromised. This increases bactericidal capacity that occurs at a reduced pH and concomitantly reduces cell function. The reduced pH unbalances signaling, further mounting an inflammatory response which results in the onset and progression of disease. Altering or manipulating the pH, cellular pHe and pHi to an optimal or near optimal range restores the electro-physical environment and provides disease resistance. Cesium and/or rubidium ions provide an “electron bath” in which the free radicals are bathed. As a result, the free electrons are stabilized and no longer able to induce cellular and tissue damage. The properly functioning electro-biological environment has a narrow pHe ranging between about 7.20 to about 7.50, and preferably between about 7.37 and about 7.42.

The current invention's compositions may be administered to reduce or eliminate pain or discomfort. The relief offered by the compositions is typically long lasting, such that the reduction or elimination in pain occurs quickly and for a substantial period of time. Treatment of pain using the compositions and methods of the present invention is not limited to humans. Domestic animals, such as dogs, cats, and horses may also be treated with the subject compositions.

The present invention encompasses administering the composition as an adjunct with surgical procedures for reducing the amount of scar tissue formation during surgical procedures. Scar tissue occurs when there is an incorporation of large quantities of damaged cells in the formation of tissues. This occurs when infected cells activate the immune system at the site of tissue damage. Cesium reduces the production of and kills/destroys the damaged and/or defective cells which results in less scarring, i.e. there are less defective and damaged cells to be enveloped and this enhances the tissues' healing process at the site of the damage.

The present invention also encompasses administering the composition of the present invention incorporated within a dressing material for the treatment of wounds by placing corpuscles on the dressing, most frequently by means of an irreversible adhesive connection, where the corpuscles contain at least one substance which is active in wound care that is stable at temperatures below 50 degrees C. One or more biologically active substances are dispersed, suspended, dissolved, or are present as a coating thereon incorporating a therapeutically effective amount of one or more therapeutically active (e.g. anti-microbial) compounds. Secondary active substances may also be used such as EGF (epidermal growth factor), EGF-urogastron, immunoglobulins, antiseptics, antibiotics, anti-inflammatory agents, proteolytics, local anesthetics, heparin and vasodilators etc.

The inventive composition encompasses all pharmaceutically active species of cesium and or rubidium, and may also encompass hydrated versions, such as aqueous solution, hydrolyzed products or ionized products, of these compounds. The compounds may contain a different number of attached water molecules. The composition includes any cesium and or rubidium salt compound(s), whether used alone or in combination with other compounds, that can alleviate, reduce, or ameliorate infections and pain.

The method and composition of the present invention encompasses administering the composition as an adjunct with low thermal laser procedures. Administration of the inventive composition as an adjunct with laser therapy reduces the scarring and adhesions that occurs as a result of wounds and/or surgical procedures and simultaneously eliminates staff infections thereby promoting and stimulating the healing process.

An exemplary embodiment of the present invention includes administering high dose or doses of the composition by slow I.V. drip simultaneously or sequentially with cesium and or rubidium chelators. The therapy comprises a treatment schedule for administering chelating agents to chelate the excess cesium and/or rubidium from the patient's body as fast as possible. Examples of cesium chelators include calcium, Omega 3 oil, cod liver oil or other fish oils (for bonding with low-density proteins). The chelation of the Vitamin E bonds with low-density lipo-proteins and omega 3 oils so that the body can expel it. Sequential administration is preferred. One example includes sequentially administering cesium and rubidium chelating agents to a 70 kg male for 24 hours including Vitamin A with doses ranging between about 2,000 IU to about 15,000 IU per 24 hours, and simultaneously or sequentially administering Vitamin E (d-alpha tocopherol) with doses ranging between about 5 IU to about 80 IU per 24 hours. Compounds intended to combat secondary infection such as antibiotics with antiviral, anti-bacterial, antifungal, and anti mold action may be included if appropriate.

When used in conjunction with the composition of the present invention, oral chelators are preferred. They are preferably administered 1 to 4 hours after initial administration, and more preferably between 2 and 4 hours after initial dose administration, regardless of route of administration. Chelators are not necessary unless more than 2000 mg of the composition of the present invention is administered per 24 hours, more than 10 grams of the composition is administered within a week, or the patient has had previous cesium or rubidium therapy. High dosage ranges should be reserved for life threatening situations/wounds.

Free cesium ions have more energy and burn less than rubidium and the ions that get formed as a factor of the cesium are short-term ions. Rubidium ions are less sight-directed ions. Cesium ions are more efficiently site directed to the affected acidotic wound site or acidotic infection site than the rubidium ions, and cesium ions are more electro-physically efficient than rubidium ions. The frequency of the cesium ions will cause damage to microorganisms having thin cell walls thereby enabling its selectivity in treating specific wounds and diseases. Cesium ions are preferred.

The method and composition of the present invention encompasses administration by direct injection into necrotized tissue (gangrene) in the vicinity of a wound with a syringe or a suitable delivery method to improve or enhance site directing or targeting. The composition contains enhanced viscosity containing a surfactant (penetrating agent) with Vitamin B12. The composition is adjusted with a surfactant to have a viscosity that preferably falls between about 8 to about 36 dynes per cm², more preferably ranging between about 10 to about 35 dynes per cm², to sufficiently penetrate into the nucleus of the infected cells without contributing to systemic toxicity or the collateral damage of healthy viable cells.

The composition may be manufactured by conventional pharmaceutical methods containing buffered salt or salts. If preferred, the active ingredients (cesium salts) may be administered without previous dissolution or they may be prepared as a solution suitable for ingestion or injection using carrier and/or surfactant liquids. For example, solutions suitable for injection should be prepared that render the solution's pH balanced and acceptable for injection. Typically, injectable solutions will be comprised of active cesium salts in a sterile buffered saline solution isotonic to blood. The I.V. injected solution should be isotonic to blood and have a physiological pH range between about 7.20 to about 7.50, preferably ranging between about 7.30 to about 7.44.

In one embodiment of the current invention, the surface tension of the composition is adjusted to a range between about 8 to about 36 dynes per cm², with an O.R.P. from about −1 mV to about −100 mV, preferably ranging between about −5 mV to about −50 mV, and most preferably about −5 mV to about −25 mV.

Method of Use

Modes of Administration

The compositions of the present invention may be administered by any suitable route including, but not limited to: atomized, periodic injections, rectal, vaginal, nasogastric, rinse, topical transdermal application, subcutaneous, reversed catheter, implanted osmotic mini-pump or continuous infusion, or other slow-release methods or devices. Topical administration and rinses are preferred.

The compounds may be formulated for administration by injection, e.g., by bolus injection or continuous infusion. Compositions for injection may be presented in unit dosage form, e.g., in ampoules or in multi-dose containers, with an added preservative if required. The compositions may take a wide variety of forms including, but not limited to, suspensions, solutions or emulsions in oily or aqueous vehicles. They also may contain formulatory agents such as suspending, stabilizing and/or dispersing agents. Alternatively, the active ingredient may be in powder form for constitution with a suitable vehicle, e.g., sterile pyrogen-free water or other solvents, before use.

The composition may take the form of gels, oils, bandages, patches, dressings, gauze, topical lotions, douche solutions, suppositories, colon irrigation rinses or solutions, drop dispersions, encapsulization in liposomes, micro-particles, enteric coatings, micro capsules, transdermal patches, etc. Compositions of the present invention suitable for administration may be presented as discrete units such as capsules, cachets or tablets each containing a predetermined dose of the active ingredients; as a powder or granules; as a solution or a suspension in an aqueous liquid or a non-aqueous liquid; or as an oil in water liquid emulsion or a water in oil liquid emulsion. The active ingredient may also be presented as a bolus, electuary or paste.

In one specific embodiment, the composition is administered two times daily for the first period of time. In another specific embodiment, the composition is administered one to two times daily for a second period of time. In yet another specific embodiment, the composition is administered one to two times daily for yet a third period of time or as needed.

In practical use, the composition can be combined as the active ingredient in intimate admixture with a pharmaceutical carrier or incipient according to conventional pharmaceutical compounding techniques. The carrier may take a variety of forms depending on the form of preparation desired for administration, e.g., oral or parenteral (including tablets, capsules, liquids, syrups, powders, intravenous injections or infusions). In preparing the compositions for oral dosage form any of the usual pharmaceutical media may be employed, e.g., water, glycols, oils, alcohols, flavoring agents, preservatives, coloring agents, and the like; in the case of oral liquid preparations, e.g., suspensions, solutions, elixirs, liposomes and aerosols; starches, sugars, micro-crystalline cellulose, diluents, granulating agents, lubricants, binders, disintegrating agents, and the like.

Treatment Protocols

One embodiment of the present invention encompasses administering the composition contained in the form of a bandage or gauze for wound treatments. For example, the composition may be administered topically with a pH ranging between about 7.00 to about 9.50, more preferably ranging between about 7.00 to about 8.50 with an oxidation reduction potential (ORP) ranging between about −1 mV to about −100 mV, preferably ranging between about −10 mV to about −50 mV. A suitable dose is administered to effect the injured sites and cells and to establish an electro-minus charge. This promotes the electrical flow which simultaneously reduces scaring, promotes anti-bacterial activity, and promotes the healing cycle.

The compounds of the current invention may be administered orally or via injection at doses ranging from about 500 to about 4000 mg per 24 hrs for a 70 kg male, preferably ranging between about 1000 to about 3000 mg per day. Doses above 1000 mg per day may be given in a series of smaller doses over a 24-hour period by continuous infusion over several hours and are preferably sequentially administered with cesium and or rubidium chelators as necessary. Preferably, the dose given will range from approximately 1000 mg to 2500 mg per 24 hours. This may equate to 2 to 3 capsules, according to the above formulation, by oral administration for one dose. The patient is given the composition two to three times daily until the healing cycle is induced in order to prevent complications due to the potential toxic accumulation of the composition in the body. Continuous oral or I.V. administration should not exceed 60 days, consisting of 5 consecutive days of administration followed by 15 consecutive days resting period subsequent to each 5 day administration cycle, for a maximum of 60 days including resting periods. Doses over 3000 mg per 24 hours should be reserved for life threatening situations.

When administering larger doses of the composition (which includes cesium salts), it is necessary to sequentially administer the composition 2 to 4 hours post-administration with chelating agents that will bond with any excess cesium that does not get site-directed to the injured or necrotized tissue. Chelators should be employed that have an affinity for the cesium or cesium salts (such as calcium or potassium, omega 3 oils, etc.) to effectively remove the non-targeted cesium from the patient system as quickly as possible. The physician should be careful to avoid administering excessive doses of potassium that will cause arrhythmic responses in the heart.

Supplementary active ingredients may also be administered such as methylcyanocobalamin—a form of Vitamin B12 that creates negative ions in the body. These negatively ionized radicals are beneficial attractors to the acidotic damage and infected cells and tissues. Methylcyanocobalamin creates twice as many negative ions as cyanocobalamin (also a form of Vitamin B12). Methylcyanocobalamin may be substituted as an alternative or as a variation of the present invention and may be administered in doses of about 50% or less than cyanocobalamin for a similar therapeutic effect. Cyanocobalamin is preferred.

Another exemplary embodiment of the present invention encompasses administering Vitamin E doses as a catalyst and a chelator (bonding agent) sequentially with the composition and therapy of the present invention. About 1000 I.U. of Vitamin E may be sequentially administered with omega 3 oils ranging between about 5000 I.U. to about 10,000 I.U. for a 70 kg male per 24 hours. Doses are preferably sequential and range between 1 to 2 hours apart from each other. Oral administration is preferred by administering the omega 3 oil with the patient's food and then administering the Vitamin E on its own.

The method and composition of the present invention also encompasses producing a topically administered composition or solution with an ORP (oxidative reduction potential) ranging between about −1 mV to about −50 mV, preferably ranging between about −1 mV to about −30 mV, with a pH between about 6.90 to about 7.80, preferably between about 7.00 to about 7.22.

The present invention also encompasses a method for reducing inflammation within a patient's body in the vicinity of infection. The method involves injecting a composition having cesium and/or rubidium ions in an amount ranging between about 1,500 ppm to about 100,000 ppm, with a preferable amount ranging from about 2,000 ppm to about 50,000 ppm.

When administering maximum doses of the composition orally or by slow I.V. drip, the composition is preferably administered with active nutrient support including administration during the resting and testing periods. Nutrient support may include, but is not limited to, Vitamin B6, Vitamin B12, and Vitamin A. Vitamin B6 (pyridoxine) is administered for detoxifying the neurological system and Vitamin B12 (cyanocobalamin) for detoxifying the bone, muscle and connective tissues. The administration of Vitamin B17 helps detoxify the endocrine system.

Another exemplary embodiment of the present invention encompasses administering Vitamin D2 and/or Vitamin D3 in conjunction with the method and composition of the present invention. An example of this embodiment includes orally administering dosages of Vitamin D2 ranging between about 1,000 I.U. to 5,000 I.U. for a 70 kg male, preferably initially administering about 1,000 I.U. per 24 hours and slowly elevating the dose, if indicated, up to and not exceeding 5,000 I.U. per 24 hrs. Stand-alone doses of Vitamin D3 about identical to the Vitamin D2 doses referenced herein may also be administered in conjunction with the method and composition of the present invention. The active nutrient support doses may be reduced each week by about one half and then further reduced each week by one half again until the dose is at the M.D.R. (minimum daily requirement). The minimum daily requirement is then maintained.

In one embodiment of the present invention, the method and composition encompasses administering compositions to treat patients suffering from bacterial or viral and/or parasitic infections leading to precancerous lesions, polyps or cancer. The present invention encompasses administering the composition in a saline solution which promotes enhanced site affinity that is suitable for repeated use. The composition's actives, i.e. cesium and/or rubidium ions, penetrate and disrupt the metabolic function of thin-walled microorganisms such as bacteria, interrupting their ability to function, grow and reproduce. Normal human cells are thick-walled, and are therefore generally unaffected by the composition's actives. The method and composition of the present invention encompasses administering a combination of different salts containing cesium and/or rubidium. Suitable rubidium or cesium salts include the citrates, bromides, sulfates, sulfides or carbonates thereof. A particular advantage associated with the use of the present composition (in a powdered or granular form) as an antibacterial agent is that it can withstand sterilization by autoclaving whereas many conventional antibacterials such as penicillin cannot. The composition of the present invention also has a long shelf life and does not require refrigeration.

In general, as a non-limiting example, the compositions of the invention when formulated with salts containing cesium and/or rubidium will be approximately isotonic and will have an osmolarity greater than about 240 mOsm/litre, e.g. in the range of about 250 to 320 mOsm/litre, for example from about 250 to 290 mOsm/litre. This osmolarity will be primarily due to electrolytes. Examples of suitable cations and anions include sodium, potassium, calcium, chloride, lactate, citrate and bicarbonate. It is possible, however, to administer via a central catheter without electrolytes, in which case the osmotic pressure would be lower, e.g. about 145 mOsm/litre.

The compositions according to the invention are of particular use as infusions, electrolyte and cesium salt or salts solutions, and as surgical rinse solutions. In particular, the compositions may be used as short-term plasma expanders and blood substitutes, for increasing the oncotic pressure in infusion solutions without any antigenic effect, and in the treatment of blood loss, sepsis and burns. Such use provides volume stability; in contrast to pure electrolyte solutions which rapidly traverse the tissue of the intravasal area. In general, microcirculation, haematocritic reading, erythrocyte deformation, erythrocyte aggregation and blood and plasma viscosity may be improved (e.g. in peripheral and arterial obstruction diseases). As a non-limiting example, the dose and rate of infusion of the compositions of the invention depends on the concentration and on the clinical situation; for example the dosage for a solution comprised of the combination of 2 cesium salts ranging from about 2% to about 5% total cesium salts is 250 ml/2 hrs drop infusion per 70 kg body weight via a central catheter.

Another exemplary embodiment of the present invention encompasses a method and composition that may be used in a wide variety of medical compositions as an admixture and still another exemplary embodiment of the present invention encompasses a method and composition as an adjunct for organ transplants. The method and composition of the present invention can be tailored/customized to the treatment of a specific individual's wound or disease by varying the elements of the composition as described herein and the administration of the composition.

The facts and theories discussed in this disclosure are intended to teach the physician how to use the invention. While this invention has been described in connection with preferred embodiments, it is obvious that various modifications, changes or substitutions therein may be made by those skilled in the art to which it pertains, without departing from the spirit and scope of the invention. Accordingly, the scope of the present invention is to be limited only by the appended claims and their legal equivalents. The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, compositions, compounds, method steps, order, sequence, structural arrangements, functions, or the like.

1. A composition for treating and healing wounds comprising:
at least one of a cesium ion source and a rubidium ion source; and
at least one surfactant capable of adjusting a surface tension of the composition within a range of about 8 dynes per cm² to about 50 dynes per cm².

2. The composition of claim 1 wherein the cesium ion source comprises a cesium salt combination of cesium sulfate and cesium citrate.

3. The composition of claim 2 wherein the cesium salt comprises a combination of cesium sulfate and cesium citrate in a ratio having a range of about 2-3 cesium sulfate to about 1 cesium citrate.

4. The composition of claim 2 wherein the cesium salt combination comprises a range of about 0.10% to about 5% of the composition.

5. The composition of claim 1 wherein the surfactant also functions as a suitable carrier for applying the composition to a wound.

6. The composition of claim 5 wherein said at least one surfactant comprises glycerin.

7. The composition of claim 6 wherein said at least one surfactant further comprises DMSO which is added to further adjust the viscosity of the composition.

8. The composition of claim 7 wherein said at least one surfactant comprises DMSO within a range of about 0.10% to 5%.

9. The composition of claim 1 wherein said at least one surfactant comprises at least one of a polysorbate, a sorbitan stearate, a sorbitan mono-oleate, a sarcosinate, a cetyl pyridinium chloride, a cetyl trimethyl ammonium bromide, a diisobutyl phenoxy ethoxy ethyldimethyl benzyl ammonium chloride, a coconut alkyl trimethyl ammonium nitrate, an alkyl sulfate, a fatty alcohol, a fatty amide, a polyhydric alcohol, a polyethylene glycol, a sodium lauryl sulfate, a sorbitan laurate, a sorbita palitate, a polyoxyethylene (20) sorbitan monolaurate, a polyoxyethylene (20) sorbitan monopalmitate, a polyoxyethylene (20) sorbitan monostearate, and a benzalkonium chloride.

10. The composition of claim 1 wherein the composition comprises a pH within a range of about 6.8 to 9.50.

11. The composition of claim 1 wherein the composition comprises an ORP within a range of about −1 m.v. to about −50 m.v.

12. The composition of claim 1 further comprising a suitable carrier for applying the composition to a wound.

13. The composition of claim 12 wherein the carrier comprises at least one of a water, a saline, a dextrose, and a biocompatible polymer.

14. The composition of claim 1 further comprising a chelating agent for chelating said at least one of a cesium ion source and a rubidium ion source.

15. The composition of claim 14 wherein the chelating agent comprises at least one of a calcium, an omega 3 oil, a cod liver oil, and another fish oil.

16. The composition of claim 1 wherein the cesium ion source comprises a cesium salt selected from at least one of a Carbonate, a Chloride, a Citrate, a Malic, a Nitrate, a Phosphate, a Sulfite, a Sulfate.

17. The composition of claim 1 further comprising a soothing agent which includes a eucalyptus, a menthol, or a mentholyptus composition.

18. The composition of claim further comprising an electrolyte comprising at least one of a sodium, a potassium, a calcium, a chlorate, a magnesium, a bicarbonate, a phosphate, and a sulfate.

19. The composition of claim 1 further comprising at least one of an antibacterial agent, an antifungal agent, and an anti mold agent.

20. The composition of claim 1 wherein said composition is incorporated into or formulated into at least one of a wet aerosol, a dry aerosol, a salve, an ointment, a cream, a liquid solvent, a nasal spray, a suppository, a gel, a solution, a suspension, an infusion, a lotion, a douche solution, a colon irrigation rinse, a surgical rinse, an oral rinse, a drop dispersion, an encapsulated microparticle, and enteric coating, a microcapsule, a transdermal patch, and a wound dressing.

21. The composition of claim 1 further comprising a nutrient support including at least one of manganese, Vitamin A, Vitamin E, Vitamin B6, Vitamin D2, Vitamin D3, and Vitamin B12.

22. The composition of claim 1 further comprising at least one of a haemostatic agent, an antiseptic agent, an anti-inflammatory agent, a proteolytic agent, and a vasodilating agent.

23. A device comprising at least one of a gauze, a bandage, or other wound care dressing which incorporates the composition of claim 1.

24. The device of claim 23 wherein said at least one of a gauze, a bandage, or other wound care dressing includes an outer fabric support and an inner pad having an outer membrane surface wherein the outer membrane surface of the inner pad is capable of having the composition of claim 1 incorporated therein.

25. A wound care kit comprising:
a package containing a composition having at least one of a cesium ion source and a rubidium ion source and at least one surfactant capable of adjusting a surface area of the composition within a range of about 8 dynes per cm² to 36 dynes per cm²; and
a bandage or other wound care dressing.

26. The wound care kit of claim 25 further comprising a material for packing a wound for use in combination with the composition and the bandage or other wound care dressing.

27. The wound care kit of claim 25 wherein the bandage or other wound care dressing comprises an oxygen-permeable material.

28. The wound care kit of claim 25 wherein the package is comprised of a material which provides a non-reactive surface with the composition.

29. A method for treating or healing a wound comprising the step of administering the composition of claim 1.

30. The method of claim 29 wherein the method for treating or healing a wound comprises a method for treating and healing at least one of a burn, a sore, an infection, an inflammation, a scar, a dermatological condition, a polyp, an adhesion resulting from surgery, a tumor, and a lesion.
